SEQUENCE LISTING (10 sequences)

SEQ ID NO: 1 - DNA, 822 bp, Mus musculus
atgcatgtaccgggcaccagagcaaagatgtccagcatctttgcctatcagagttctgaggtggattggtgtgagagtaatttccagcactcagagttggtggccgagttctacaatacgttcagcaatgtgttcttcctcatctttggacccctcatgatgttcctcatgcatccgtatgcccagaagcgtacccggtgtttctatggagtgtcagtcctcttcatgctcataggtctgttctccatgtatttccacatgacactcagctttctgggacagctgctggatgagatctccatcctgtggttgttggccagtggatacagtgtgtggctgccccgttgctattttcccaagttcgtcaaggggaacaggttctacttcagctgcctggtaactataaccactattatcagcaccttcttgacgttcgtgaagcccacagtcaatgcatatgctctcaacagcatcgccatccacatcctctacattgtgcgcacagagtacaagaagattagggatgatgatcttcggcatctgattgcggtttctgtggtcttatgggccgctgcactgaccagctggatcagtgaccgtgtactttgcagcttctggcagcggattcacttctactacctgcacagcatttggcacgtcctcataagcatcacatttccttatggtatcgtgaccatggccctggtggatgcaaagtatgagatgccagataaaaccctcaaagtccactactggccccgggacagctgggtcatcgggctaccctatgtggagatccaggagaatgacaagaactgctga

SEQ ID NO: 2 - DNA, 795 bp, Homo sapiens
atgcctagcatcttcgcctatcagagctccgaggtggactggtgtgagagcaacttccagtactcggagctggtggccgagttctacaacacgttctccaatatccccttcttcatcttcgggccactgatgatgctcctgatgcacccgtatgcccagaagcgctcccgctacatttacgttgtctgggtcctcttcatgatcataggcctgttctccatgtatttccacatgacgctcagcttcctgggccagctgctggacgagatcgccatcctgtggctcctgggcagtggctatagcatatggatgccccgctgctatttcccctccttccttggggggaacaggtcccagttcatccgcctggtcttcatcaccactgtggtcagcacccttctgtccttcctgcggcccacggtcaacgcctacgccctcaacagcattgccctgcacattctctacatcgtgtgccaggagtacaggaagaccagcaataaggagcttcggcacctgattgaggtctccgtggttttatgggctgttgctctgaccagctggatcagtgaccgtctgctttgcagcttctggcagaggattcatttcttctatctgcacagcatctggcatgtgctcatcagcatcaccttcccttatggcatggtcaccatggccttggtggatgccaactatgagatgccaggtgaaaccctcaaagtccgctactggcctcgggacagttggcccgtggggctgccctacgtggaaatccggggtgatgacaaggactgctga

SEQ ID NO: 3 - DNA, 26 bp, Artificial Sequence
  Synthesized
gataccataaggaaatgtgatgctta

SEQ ID NO: 4 - DNA, 26 bp, Artificial Sequence
  Synthesized
atccctaatcttcttgtactctgtgc

SEQ ID NO: 5 - DNA, 28 bp, Artificial Sequence
  Synthesized
gcggatccatgcatgtaccgggcaccag

SEQ ID NO: 6 - DNA, 33 bp, Artificial Sequence
  Synthesized
gcgaattctcagcagttcttgtcattctcctgg

SEQ ID NO: 7 - DNA, 24 bp, Artificial Sequence
  Synthesized
agttctgaggtggattggtgtgag

SEQ ID NO: 8 - DNA, 24 bp, Artificial Sequence
  Synthesized
tggactttgagggttttatctggc

SEQ ID NO: 9 - DNA, 22 bp, Artificial Sequence
  Synthesized
tgtgatggtgggaatgggtcag

SEQ ID NO: 10 - DNA, 22 bp, Artificial Sequence
  Synthesized
tttgatgtcacgcacgatttcc

(19) United States
(12) Patent Application Publication
(10) Pub. No.: US 2006/0099681 A1
(43) Pub. Date: May 11, 2006

(54) Human and mouse alkaline ceramidase 1 and skin diseases

(75) Inventors: Lina M. Obeid, Sullivan's Island, SC (US); Cungui Mao, Mt. Pleasant, SC (US); Ruijuan Xu, Mt. Pleasant, SC (US)

Correspondence Address: DINESH AGARWAL, P.C., 5350 SHAWNEE ROAD, SUITE 330, ALEXANDRIA, VA, 22312, US

(73) Assignee: U.S. DEPARTMENT OF VETERANS AFFAIRS

(21) Appl. No.: 11/194,699

(22) Filed: Aug. 2, 2005

Related U.S. Application Data
(60) Provisional application No. 60/600,797, filed on Aug. 12, 2004.

(51) Int. Cl. C07H 21/04; C12P 21/06; C12N 9/24; C12N 5/06

(52) U.S. Cl. 435/69.1; 435/200; 435/320.1; 435/354; 536/23.2

(57) ABSTRACT

Newly discovered, isolated, and cloned human alkaline ceramidase 1 (haCER1) and mouse alkaline ceramidase 1 (maCER1) are provided, which are predominantly expressed in skin cells and hydrolyze D-erthyro-ceramide exclusively.

### CROSS-REFERENCE TO RELATED APPLICATIONS

The present application claims priority on prior U.S. Provisional Application Ser. No. 60/600,797, filed Aug. 12, 2004, which is hereby incorporated herein in its entirety by reference.

### STATEMENT REGARDING FEDERALLY SPONSORED RESEARCH OR DEVELOPMENT

The work leading to the present invention was supported by one or more grants from the U.S. Government, including NIH Grant(s): GM62887-01 and 1P20RR17677-01, and Veterans Affairs merit award. The U.S. Government therefore has certain rights in the invention.

### REFERENCE TO SEQUENCE LISTING

The present application incorporates by reference a file named: US 1438/05 Obeid Sequence Listing, including SEQ ID NO.: 1, SEQ ID NO.: 2, SEQ ID NO.: 3, SEQ ID NO.: 4, SEQ ID NO.: 5, SEQ ID NO.: 6, SEQ ID NO.: 7, SEQ ID NO.: 8, SEQ ID NO.: 9 and SEQ ID NO.: 10, provided herewith in a computer readable form—on a diskette, created on Jul. 26, 2005 and containing 8,677 bytes. The sequence listing information recorded on the diskette is identical to the written (on paper) sequence listing provided herein.

### BACKGROUND OF THE INVENTION

The present invention is directed to identification, isolation, and cloning of a mouse alkaline ceramidase (maCER1) and a human alkaline ceramidase (haCER1).

Both ceramide (phytoceramide-PHC) and sphingosine (phytosphingosine-PHS) have been shown to induce growth arrest and apoptosis of mammalian cells, whereas sphingosine-1-P (S1P) appears to promote cell growth and proliferation, and suppress apoptosis (References 1-3). As a result of the opposing effects of ceramide and sphingosine versus S1P, their relative levels may regulate the ability of cells to grow, to survive, or to die. Ceramidases are enzymes that break down the amide linkage of ceramides (ceramide, dihydroceramide, and phytoceramide) to generate free fatty acids and sphingoid bases (sphingosine, dihydrosphingosine, or phytosphingosine) (Reference 4), which in turn are phosphorylated by sphingosine kinases to generate sphingoid base phosphates (References 2,3 and 5). Since ceramidases are capable of regulating the levels of these bioactive lipids, they may have an important role in regulating cellular responses mediated by these lipids. Furthermore, ceramides are also the building blocks of complex sphingolipids which have important structural and functional roles (References 6 and 7)). Therefore, ceramidases may also regulate sphingolipid-mediated biology by regulating metabolism of the precursors of complex sphingolipids.

Ceramidases, according to their pH optima for activity, fall into three groups-acid, neutral, and alkaline (Reference 4). The human acid ceramidase is a lysosomal enzyme (Reference 8), whose inherited deficiency leads to the lipid-storage disease, Farber disease (Reference 9). Deletion of the mouse acid ceramidase leads to embryonic lethality (Reference 10), suggesting that this enzyme is indispensable for the development of mouse embryos.

Neutral (non-lysosomal) ceramidases were cloned from pseudomonas (Reference 11), mouse (Reference 12), rat (Reference 13), and humans (Reference 14). In vitro, these ceramidases appear to have both ceramidase activity and (CoA-independent) ceramide synthase activity (References 15-17). The human enzyme was shown to be localized in mitochondria (Reference 14), or extracellularly secreted from endothelial cells (Reference 18), whereas the rat enzyme was found in hepatocyte endosomes and intestinal apical membrane (Reference 13). Their physiological roles are still unclear.

Alkaline ceramidases (YPC1p and YDC1P) were first cloned from the yeast Saccharomyces cerevisiae in our laboratory (References 19 and 20). YPC1p and YDC1p share a 50% protein identity, are localized in the endoplasmic reticulum (ER), and have an alkaline pH optimum, but differ in some biochemical properties. YPC1p exhibits both ceramidase and (CoA-independent) ceramide synthase activities in vitro, and mainly hydrolyzes phytoceramide. YDC1p has a major ceramidase activity but a minor ceramide synthase activity in vitro, and hydrolyzes dihydroceramide preferentially.

Based on sequence similarity to YPC1p and YDC1p, we recently identified and cloned a human alkaline phytoceramidase (haPHC) that preferentially hydrolyzes phytoceramide (Reference 21), and exhibits no ceramide synthase activity in vitro. haPHC is localized to both the Golgi and ER.

These alkaline ceramidases appear to have roles in regulating metabolism of sphingolipids and modulating biologic responses. Deletion or overexpression of the yeast alkaline ceramidases significantly alters the turnover of complex sphingolipids, free sphingoid bases, and their phosphates (References 19 and 20). In addition, deletion of YDC1p reduces the tolerance of yeast cells to heat stress (Reference 19). Overexpression of haPHC in yeast cells leads to increased hydrolysis of phytoceramide and a concomitant increase in the generation of phytosphingosine and its phosphate (Reference 21), and leads to suppression of yeast cell growth.

We demonstrate that maCER1 and haCER1 exclusively hydrolyze D-erythro-ceramide, and possess the ability to regulate the levels of very long chain ceramides, S1P, and complex sphingolipids. There enzymes are localized in the endoplasmic reticulum (ER). The identification of these alkaline ceramidases reveals that turnover of ceramides is carried out by multiple distinct ceramidases in different compartments, suggesting complexities in regulating the levels of ceramide, sphingosine, and sphingosine-1-P in mammalian cells.

### OBJECTS AND SUMMARY OF THE INVENTION

An object of the present invention is to provide an isolated mouse alkaline ceramidase (maCER1).

Another object of the present invention is to provide a cloned mouse alkaline ceramidase (maCER1).

Another object of the present invention is to provide a newly discovered mouse alkaline ceramidase (maCER1) and its coding nucleotide sequence, which have therapeutic, diagnostic, nonmedical, and/or other utilities.

Yet another object of the present invention is to provide mouse alkaline ceramidase 1 that hydrolyzes D-erythro-ceramide exclusively.

Still yet another object of the present invention is to provide an isolated nucleotide sequence as set forth in SEQ ID NO: 1.

Still yet another object of the present invention is to provide a cloned nucleotide sequence as set forth in SEQ ID NO: 1.

Still yet another object of the present invention is to provide a nucleotide sequence encoding mouse alkaline ceramidase 1.

An additional object of the present invention is to provide an isolated human alkaline ceramidase (haCER1).

An additional object of the present invention is to provide a cloned human alkaline ceramidase (haCER1).

Yet an additional object of the present invention is to provide a newly discovered human alkaline ceramidase (haCER1) and its coding nucleotide sequence, which have therapeutic, diagnostic, nonmedical, and/or other utilities.

Yet an additional object of the present invention is to provide human alkaline ceramidase 1 that hydrolyzes D-erythro-ceramide exclusively.

Still yet an additional object of the present invention is to provide an isolated nucleotide sequence as set forth SEQ ID NO: 2.

Still yet an additional object of the present invention is to provide a cloned nucleotide sequence as set forth SEQ ID NO: 2.

Still yet an additional object of the present invention is to provide a nucleotide sequence encoding human alkaline ceramidase 1.

In summary, the main object of the present invention is to provide newly identified, isolated, and cloned human alkaline ceramidase 1 and mouse alkaline ceramidase 1 that hydrolyze D-erythro-ceramide exclusively.

Ceramidases deacylate ceramides, important intermediates in the metabolic pathway of sphingolipids. In this invention, we report the cloning and characterization of a novel mouse alkaline ceramidase (maCER1) with a highly restricted substrate specificity. maCER1 consists of 287 amino acids and it has a 28% and 32% identity to the Saccharomyces alkaline ceramidases (YPC1p and YDC1p) and the human alkaline phytoceramidase (haPHC), respectively. RT-PCR analysis demonstrated that maCER1 was predominantly expressed in skin. maCER1 was localized to the endoplasmic reticulum as revealed by immunocytochemistry. In vitro biochemical characterization determined that maCER1 hydrolyzed D-erythro-ceramide exclusively, but not D-erythro-dihydroceramide or D-ribo-phytoceramide. Similar to other alkaline ceramidases, maCER1 had an alkaline pH optimum of 8.0, and it was activated by Ca²⁺, but inhibited by Zn²⁺, Cu²⁺, and Mn²⁺. maCER1 was also inhibited by sphingosine, one of its products. Metabolic labeling studies showed that overexpression of maCER1 caused a decrease in the incorporation of radiolabeled dihydrosphingosine (DHS) into ceramide and complex sphingolipids, but led to a concomitant increase in S1P in Hela cells. Mass measurement showed that overexpression of maCER1 selectively lowered the cellular levels of D-erythro-C_{24:1}-ceramide, but not other ceramide species, and caused an increase in the levels of S1P. Our data support the conclusion that maCER1 is a new alkaline ceramidase with a stringent substrate specificity, and that maCER1 is selectively expressed in skin and regulates the levels of bioactive lipids ceramide and S1P as well as complex sphingolipids.

One of the above objects is met, in part, by the present invention which in one aspect includes an isolated nucleotide sequence as set forth in SEQ ID NO: 1.

Another aspect of the present invention includes a cloned nucleotide sequence as set forth in SEQ ID NO: 1.

Another aspect of the present invention includes a nucleotide sequence encoding mouse alkaline ceramidase 1.

Another aspect of the present invention includes a substantially purified isolated nucleotide sequence encoding a mouse alkaline ceramidase having the ability to hydrolyze mammalian D-erythro-ceramide.

Another aspect of the present invention includes an isolated mouse alkaline ceramidase having the ability to hydrolyze mammalian D-erythro-ceramide.

Another aspect of the present invention includes a nonnaturally occurring analogue of the mouse alkaline ceramidase 1 (maCER1).

Another aspect of the present invention includes a recombinant mouse alkaline ceramidase having the ability to hydrolyze mammalian D-erythro-ceramide.

Another aspect of the present invention includes an isolated nucleotide sequence-deposited with GenBANK (www.ncbi.nlm.nih.gov) with an Accession Number AF 347023.

Another aspect of the present invention includes an isolated nucleotide sequence as set forth in SEQ ID NO: 2.

Another aspect of the present invention includes a cloned nucleotide sequence as set forth in SEQ ID NO: 2.

Another aspect of the present invention includes a nucleotide sequence encoding human alkaline ceramidase 1.

Another aspect of the present invention includes a substantially purified isolated nucleotide sequence encoding a human alkaline ceramidase having the ability to hydrolyze mammalian D-erythro-ceramide.

Another aspect of the present invention includes an isolated human alkaline ceramidase having the ability to hydrolyze mammalian D-erythro-ceramide.

Another aspect of the present invention includes a recombinant human alkaline ceramidase having the ability to hydrolyze mammalian D-erythro-ceramide.

Another aspect of the present invention includes a nonnaturally occurring analogue of the human alkaline ceramidase 1 (haCER1).

Another aspect of the present invention includes an isolated nucleotide sequence deposited with GenBank (www.ncbi.nlm.nih.gov), with an Accession Number AF 347024.

Another aspect of the present invention includes a recombinant protein sequence deposited with GenBank (www.ncbi.nlm.nih.gov) with an Accession Number AAL83821.

Another aspect of the present invention includes a recombinant protein sequence deposited with GenBank (www.ncbi.nlm.nih.gov) with an Accession Number AAL83822.

### BRIEF DESCRIPTION OF THE DRAWINGS

One of the above and other objects, novel features and advantages of the present invention will become apparent from the following detailed description of the preferred embodiment of the invention, as illustrated in the drawings, in which:

FIGS. 1A-B illustrate cDNA and protein sequences of the mouse alkaline ceramidase 1 and the sequence alignment of alkaline ceramidases. FIG. 1A-the mouse alkaline ceramidase (maCER1) coding sequence preceded by 5′-UTR was cloned by RACE as described under “Experimental Procedures”. The protein sequence was deduced from the DNA sequence by the MacVector software. Underlined and starred is the in-frame stop codon upstream of the putative translational initiation site. Putative transmembrane domains (underlined) and a Golgi-ER retrieval sequence (dashed line) were predicted using the pSORTII program. FIG. 1B-protein sequences were aligned using the ClustalW method. Identical amino acids across all aligned proteins are depicted in yellow, identical amino acids among two or three proteins in blue, and similar amino acids in green. Underlined are conserved regions among all enzymes.

FIGS. 2A-D illustrate overexpression of maCER1 imparts a ceramidase activity to the yeast mutant Δypc1Δydc1 devoid of ceramidase activity. Microsomes were prepared from the yeast mutant strain (Δypc1Δydc1) containing the empty vector pYES2-FLAG (Vec) or expressing the FLAG tagged maCER1 (maCER1). Proteins (40 μg) extracted from the microsomes by 0.5% Triton X-100 were subjected to Western blot analysis using the anti-FLAG antibody as described under “Experimental Procedures” (FIG. 2A). The microsomes (20 μg proteins per reaction) were assayed for ceramidase activity using 200 μM D-e-C₁₂-NBD-ceramide (N-4-nitrobenz-2-oxa-1,3-diazole) (D-e-CER), dihydroceramide (DHC), D-ribo-phytoceramide (PHC), or NBD-L-t-ceramide (L-t-CER) as substrates as described under “Experimental Procedures”. The released product, C₁₂-NBD-fatty acid, was resolved by TLC, detected by PhosphorImager under the blue fluorescent mode (FIG. 2B), and quantified by the ImageQuant software (FIG. 2C). The maCER1-containing microsomes were assayed for ceramidase activity in the presence of D-e-NBD-ceramide and its analogs at different concentrations (FIG. 2D). Results are the mean±SD of three independent experiments performed in duplicate.

FIGS. 3A-C illustrate overexpression of maCER1 elevates ceramidase activity in mammalian cells. pcDNA3.1-FLAG and pcDNA3.1-FLAG-maCER1 were transfected, respectively, into COS1 cells. Forty-eight hours after transfection, microsomes were prepared from the cells. A portion (40 μg proteins per lane) of the microsomes was subjected to Western blot analysis using the anti-FLAG antibody (1:500) (FIG. 3A). Another portion (20 μg proteins per reaction) was assayed for ceramidase activity using C₁₂-D-e-NBD-ceramide (200 μM) as substrate. The released C₁₂-NBD-fatty acid (NBD-FA) was resolved by TLC and detected by the PhosphorImager (FIG. 3B). The activity was determined by the ImageQuant (FIG. 3C). Vec, vector-transfected cells; and maCER1, maCER1-transfected cells. Results are the mean±SD of three independent experiments performed in duplicate.

FIGS. 4A-C illustrate maCER1 has an alkaline pH optimum and exhibits novel biochemical properties. The microsomes, as in FIGS. 2A-D were assayed for ceramidase activity using D-e-C₁₂-NBD-ceramide (200 μM) as substrate at different pH in the presence of 0.2% NP-40 and 5 mM Ca²⁺ (FIG. 4A), at pH 8.5 in the presence of different cations (FIG. 4B), or different sphingoid bases (FIG. 4C). Results are the mean±SD of three independent experiments performed in duplicate.

FIG. 5 illustrates that maCER1 mRNA is highly expressed in skin. RT-PCR analysis for maCER1 mRNA (the upper panel) or actin mRNA (the lower panel) was performed on RNA isolated from different mouse organs as described under “Experimental Procedures”.

FIGS. 6A-B are photomicrographs showing maCER1 is mainly localized to the ER. Hela cells stably expressing the vector (Vec) or the FLAG-tagged maCER1 (maCER1) were homogenized by sonication. Cell lysates were fractionated as described under “Experimental Procedures”. Forty μg proteins extracted from the 100 k g membrane fraction were subjected to Western blot analysis using the anti-FLAG antibody (FIG. 6A). The maCER1 cell line was transfected with pEGFP-ER and subjected to immunostaining and microscopic analysis (FIG. 6B) as described under “Experimental Procedures”. As a negative control, the Vec cell line (FLAG) was also subjected to immunostaining analysis.

FIGS. 7A-D illustrate overexpression of maCER1 alters metabolism of sphingolipids. Ceramidase activity of the Vec and maCER1 cell lines were determined by the release of C₁₂-NBD-fatty acid from NBD-D-e-C₁₂-ceramide. FIG. 7A-TLC analysis of NBD-fatty acid production, and FIG. 7B-quantification of ceramidase activity. Cells with a 90% confluence in 65-mm culture dishes were labeled with [³H]-DHS (2.5 μCi per dish) overnight (14 hours) to equilibrium. Total lipids were extracted as described under “Experimental Procedures”. The lipids were resolved by TLC using a solvent of chloroform:methanol: 15 mM CaCl (65:35:8), and detected by autoradiography (FIG. 7C). The labeled sphingolipids were identified according to known standards. Radioactive lipid bands were scraped from TLC and quantified by scintillation counting (FIG. 7D). Cer, ceramide; Glu-cer, glucosylceramide; and SM, sphingomyelin.

FIGS. 8A-C illustrate overexpression of maCER1 causes a decrease in the cellular levels of ceramides with very long acyl chains, but an increase in the levels of S1P. Total lipids were extracted from the Vec and maCER1 cells grown in DMEM medium supplemented with 10% FBS, respectively. The extracted lipids were subjected to ESI/MS/MS analysis as described under “Experimental Procedures”. The levels of ceramides (FIG. 8A), sphingosine (FIG. 8B), and S1P (FIG. 8C) in the Vec and maCER1 cells were determined. Results are the mean±SD of three independent experiments performed in duplicate.

FIGS. 9A-B illustrate the mouse alkaline ceramidase 1 (maCER1) cDNA and its encoded protein sequence.

FIGS. 10A-B illustrate the human alkaline ceramidase 1 (haCER1) cDNA and its encoded protein sequence.

### DETAILED DESCRIPTION OF THE PREFERRED EMBODIMENT(S)OF THE INVENTION

The present invention relates to the newly identified or discovered, isolated, and cloned human alkaline ceramidase 1 (haCER1) and mouse alkaline ceramidase 1 (maCER1) that hydrolyze D-erythro-ceramides exclusively. Both haCER1 and maCER1 are predominantly expressed in skin, and play a critical role in regulating metabolism of ceramides and other sphingolipids in skin, and in regulating proliferation, growth, differentiation, and senescence of epidermal keratinocytes as well as the skin permeability barrier. Therefore, haCER1 and maCER1 would have important utility in serving as drug targets for skin care and diseases related to abnormality in metabolism of sphingolipids, such as atopic dermatitis, atopic eczema, psoriasis, allergy, bacterial infection, skin photoaging, skin regeneration, and skin cancers. The invention would further have utility in using the haCER1 and maCER1 polypeptides and their coding polynucleotide sequences as targets for diagnosis and treatment in skin disorders. In addition, the invention would be useful for devising drug-screening methods using the polypeptides and polynucleotides of haCER1 and maCER1 to identify inhibitors and activators of the haCER1 and maCER1 enzymes, regulators of the gene expression of haCER1 and maCER1, any other genes and proteins that interact with haCER1 and maCER1 for diagnosis and treatment of skin diseases and cancer. The invention would further be useful for producing the haCER1 and maCER1 polypeptides and polynucleotides.

### Experimental Procedures

Tissue Culture and Cell Transfection

Hela and COS1 cell lines purchased from ATCC were cultured in Dulbecco's modified Eagle's minimum (DMEM) medium supplemented with 10% fetal bovine serum (FBS), 100 units/ml penicillin G, and 100 μg/ml streptomycin at 37° C. in a humidified atmosphere of 5% CO2 and 95% air. DMEM, FBS, trypsin-EDTA, phosphate buffered saline (PBS), penicillin/streptomycin were purchased from Invitrogen, Inc.

Lipid Preparation

Glucosylceramide and lactosylceramide were purchased from Sigma. [¹⁴C]-Sphingomyelin (SM) was synthesized as described in this laboratory (Reference 22). D-erythro-sphingosine, L-threonine-sphingosine, D-erythro-dihydrosphingosine, and D-ribo-phytosphingosine were purchased from Avanti Polar-Lipids, Inc. D-erythro-C₁₂-NBD-4,5-dihydroceramide, D-ribo-C₁₂-NBD-phytoceramide, D-erythro-C₁₂-NBD-ceramide, and L-threonine-C₁₂-ceramide were synthesized as described (Reference 23). [³H]-palmitic acid was purchased from Amersham, Inc. D-erythro-4,5-[³H]-dihydrosphingosine, D-erythro-4,5-[³H]-dihydrosphingosine-1-P, D-erythro-3-[³H]-sphingosine, and D-erythro-3-[³H]-sphingosine-1-P were purchased from American Radiolabeled Chemicals.

cDNA Cloning

A search of expressed sequence tag (EST) databases in the NCBI GenBank using haPHC as a query revealed a mouse EST sequence with a putative open reading frame (ORF) that encodes a polypeptide homologous to haPHC. To clone the 5′-upstream sequence of the putative ORF, a rapid amplification of cDNA ends (RACE) was performed on a mouse liver cDNA library (Clontech, Inc.) using the RACE adaptor primer AP1 (Clontech, Inc.) and a gene specific primer (5′-GATACCATMGGAAATGTGATGCTTA-3′) (SEQ ID NO: 3) according to the manufacturer's instructions. The first-round PCR products were diluted and subjected to a second round PCR using the RACE adaptor primer AP2 and a nested gene specific primer (5′-ATCCCTAATCTTCTTGTACTCTGTGC-3′) (SEQ ID NO: 4). The resultant PCR product was cloned into a vector pCR2.1 (Invitrogen, Inc.) and sequenced.

Plasmid Construction

To construct a yeast expression vector with an epitope tag, the sense and antisense oligonucleotides of the FLAG-coding sequence flanked by Hind III and BamH I sites at 5′ and 3′-end, respectively, were synthesized. These two complementary oligonucleotides were annealed to form a double stranded DNA fragment which was cloned into the Hind III and BamH I sites of the vector pYES2 (Invitrogen, Inc.). The resulting vector pYES2-FLAG was sequenced to verify the insertion of the correct FLAG coding sequence. The ORF of the mouse alkaline ceramidase was amplified from the mouse liver cDNA library using the forward primer 5′-GC\ddot{GGATCC}ATGCATGTACCGGGCACCAG-3′ (underlined is the BamH I site) (SEQ ID NO: 5) and the reverse primer 5′-GC\ddot{GAATTC}TCAGCAGTTCTTGTCATTCTCCTGG-3′ (underlined is the EcoR I site) (SEQ ID NO: 6) as described (Reference 21). This coding sequence was cloned in frame with the FLAG tag into the BamH I and EcoR I sites of pYES2-FLAG using standard DNA cloning procedures. The resulting construct pYES2-FLAG-maCER1 was verified by sequencing. pYES2-FLAG-maCER1 was digested with the restriction enzymes Hind III and EcoR I to release the Hind III-EcoR I fragment containing the coding sequence of the FLAG-tagged maCER1. The released fragment was cloned into the compatible sites of pcDNA3.1(+) to generate the construct pcDNA3.1(+)-FLAG-maCER1, which directs expression of the FLAG-tagged maCER1 in mammalian cells.

Stable Cell Lines

At 80% confluence, Hela cells were transfected with the vector pcDNA3.1(+)-FLAG or the construct pcDNA 3.1(+)-FLAG-maCER1 using Lipofectamine 2000 (Invitrogen, Inc.) as described previously (Reference 21). Forty-eight hours after transfection, the cells from a 65-mm culture dish were dislodged by trypsin-EDTA treatment and plated into ten 10-cm culture dishes. The cells were grown in DMEM medium containing 1 g/ml G418 (high G418 medium). The high G418 medium was changed every 3-4 days until single clones were formed. The G418 resistant clones were expanded in DMEM medium in the presence of G418 (200 μg/ml) (low G418 medium) and were screened for expression of the FLAG-tagged maCER1 by both in vitro activity assay and Western blot analysis using anti-FLAG antibody. The cell lines stably expressing the FLAG tagged maCER1 were maintained in the low G418 medium. As a negative control, G418 resistant clones transfected with the empty vector pcDNA3.1(+)-FLAG were selected in the high G418 medium and maintained in the low G418 medium.

Northern Blot Analysis

Northern blot analysis was performed as described (Reference 21). Briefly, the maCER1 coding sequence was amplified by PCR from the pYES2-FLAG-maCER1, purified by gel extraction, and radiolabeled by [³²P]-dCTP using a random priming kit (Amersham). This radiolabeled DNA probe was hybridized to a multiple mouse tissue mRNA blot (Clontech, Inc.). After being washed, the hybridized membrane blot was exposed to an X-ray film (BioMax MR, Kodak) at −70° C. for one week. The same blot was hybridized by a radioactive probe for β-actin mRNA after the maCER1 probe was stripped from the blot. The membrane blot was exposed to an X-ray film for 5 hours.

Reverse Transcription DNA Polymerase Chain Reaction (RT-PCR) Analysis

Total RNA was isolated from mouse tissues using Trizol (Invitrogen, Inc.) according to the manufacturer's instructions. The isolated RNA (1 μg from each sample tissue) was reversely transcribed to cDNA by an AMV reverse transcriptase (Invitrogen, Inc.) according to the manufacturer's instructions. The resulting cDNA was subjected to PCR amplifications using Precision Tag polymerase (Stratagene, Inc.) and a maCER1 primer pair (5′-AGTTCTGAGGTGGATTGGTGTGAG-3′-SEQ ID NO: 7- and 5′-TGGACTTTGAGGGTTTTATCTGGC-3′-SEQ ID NO: 8- or β-actin primer pair (5′-TGTGATGGTGGGAATGGGTCAG-3′-SEQ ID NO: 9- and 5′-TTTGATGTCACGCACGATTTCC-3′-SEQ ID NO: 10). Conditions for the maCER1 PCR analysis were 1 cycle of 94° C. for 30 sec., 30 cycles of 94° C. for 15 sec, 58° C. for 25 sec, and 72° C. for 60 sec. The PCR product (698 base pairs) was verified by DNA sequencing. For the β-actin PCR analysis, 25 above PCR cycles were performed using one-third amount of the cDNA templates.

maCER1 Expression in Yeast Cells

maCER1 was expressed in yeast cells as described (Reference 21). Briefly, the expression construct pYES2-FLAG-maCER1 or the empty vector pYES2-FLAG was transformed into the yeast strain Δypc1Δydc1. Expression of the FLAG-tagged maCER1 was induced by 2% galactose.

maCER1 Expression in Mammalian Cells

COS1 cells were transfected with pcDNA3.1(+)-FLAG and pcDNA3.1 (+)-FLAG-maCER1 using Lipofectamine 2000 as described (Reference 21). Forty-eight hours after transfection, the cells were harvested after trypsin-EDTA treatment. After being washed three times with PBS, the cells were resuspended in buffer A (25 mM Tris-HCl, pH 7.4, containing 0.25 M sucrose, 1 mM EDTA, and 20 μg/ml protease inhibitor mixture (CLAP, Roche, Inc.)), and were sonicated for 10 seconds at a power level of 1.5 on a microtip-equipped Sonic Dismembrator (Fisher Scientific), and then chilled on ice for 30 seconds. The process of sonication and chilling was repeated twice. The total cell lysates were centrifuged at 1,000 g for 5 min. The post-nuclear supernatants were centrifuged for 10 min at 10,000 g to obtain the 10K membrane fraction. The resulting supernatant was centrifuged for 1 hour at 100,000 g to obtain microsomes (the 100K membrane fraction), which were washed three times with buffer A.

Western Blot Analysis

Proteins extracted from membranes of yeast or mammalian cells by buffer A with 0.5% Triton X-100 were resolved by SDS-PAGE (polyacrylamide gel electrophoresis), and then subjected to Western blot analysis using anti-FLAG antibody as described (Reference 21).

Immunohistochemistry

Hela cell lines containing the vector (Vec) or stably expressing the FLAG-tagged maCER1 (maCER1) were plated into 6-well plates. At 80% confluence, the cells were transfected with pEGFP-ER (Clontech, Inc.) that expresses a green fluorescent protein, which was specifically targeted to the ER. Thirty six hours after transfection, the cells were fixed by 3.7% paraformaldehyde in phosphate buffered saline (PBS) for 10 min, washed twice by PBS, and subjected to immuno-staining with anti-FLAG antibody (1:250) followed by a goat anti-mouse IgG conjugated with rhodamine (1:250) as described (Reference 21). The cells were examined under a Nikon fluorescent microscopy equipped with a digital camera.

Ceramidase Activity Assay

Ceramidase activity was determined by the release of NBD-fatty acid from fluorescent substrates, NBD-C₁₂-ceramide, dihydroceramide, or phytoceramide as described (Reference 21). Briefly, 20 μl of microsomes (containing 10-20 μg proteins) in buffer B (25 mM Tris-HCl, pH 8.0, 5 mM CaCl₂) was mixed with 20 μl of 200 μM NBD-ceramide in buffer B with 0.4% NP-40 in a 1.5 ml microfuge tube. After incubation at 37° C. for 30-60 min, the reactions were stopped by boiling for 5 min, and dried under a SpeedVec. The released NBD-fatty acid was separated from NBD-ceramide by TLC in a solvent of chloroform:methanol: ammonium hydroxide (90:30:0.5) and quantified by PhosphorImager set at the fluorescent mode. To determine the pH optimum of maCER1, the maCER1 microsomes were resuspended in buffer C (1 mM Tris-HCl, pH 7.4, containing 5 mM CaCl₂). To determine a cation effect, the maCER1 microsomes were resuspended in buffer A without CaCl₂.

Ceramidase Reverse Activity Assay

The reverse ceramidase activity was determined by the formation of ceramide (dihydroceramide, or phytoceramide) from [³H]-palmitic acid and sphingosine, dihydrosphingosine, or phytosphingosine as described (Reference 21). Briefly, 20 μl of the Vec or maCER1 microsomes (containing 20 μg proteins) in buffer A were mixed with 20 μl of substrates (100 μM [³H]-palmitic acid and 100 μM sphingosine, dihdyrosphingosine, or phytosphingosine) in buffer A, and incubated at 37° C. for 12 hours. The reactions were stopped by boiling for 5 min and dried under the SpeedVec. The [3H]-ceramide formed from the condensation of [³H]-palmitic acid and a sphingoid base was analyzed by TLC and quantified by scintillation counting.

Protein Concentration Determination

Protein concentrations were determined using a Bradford reagent (Bio-Rad) with BSA as a standard.

Sphingolipid Labeling

At 85% confluence, Hela cells grown in a 65-mm culture dish were labeled with 2.5 μCi [³H]-DHS for 14 hours. After being washed twice with PBS, the cells were scraped and subjected to lipid extraction with a solvent of chloroform:methanol: water: pyridine (60:30:6:1). After being dried under a N₂ evaporator, the extracted lipids were resolved by TLC (thin liquid chromatography) in a solvent of chloroform:methanol: 15 mM CaCl₂ (60:35:8) and detected by autoradiography as described (Reference 21). Radiolabled SPH, ceramide, S1P, and sphingomyelin were identified by their radioactive corresponding standards resolved on the same TLC plate. To visualize glucosylceramide and lactosylceramide standards, TLC plates were sprayed with a solution containing 8% (wt/vol) H₃PO₄ and 10% (wt/vol) CuSO₄ charred at 180° C. for 10 min as described (Reference 24). The radioactive lipid bands were scraped off the TLC plates and quantified by liquid scintillation counting.

ESI/MS/MS Lipid Analysis

Analysis of sphingolipids was performed on a Thermo Finnigan TSQ 7000 triple quadrupole mass spectrometer, operating in a Multiple Reaction Monitoring (MRM) positive ionization mode. Total cells, fortified with a set of internal standards, were extracted with ethyl acetate/iso-propanol/water (60/30/10 v/v). The lipid extracts were dried under a N2 evaporator, and reconstituted in 100 μl of methanol. The reconstituted samples were injected into the Survayor/TSQ 7000 LC/MS system with the BDS Hypersil C8, 150×3.2 mm, 3 μm particle size column, which was eluted with 1.0 mM methanolic ammonium formate/2 mM aqueous ammonium formate mobile phase system. The peaks for the target lipids and internal standards will be collected and processed using Xcalibur software system. Calibration curves will be constructed by plotting peak area ratios of the target lipids to their respective internal standard against concentration, using linear regression model.

### Results

Cloning of a Murine Alkaline Ceramidase

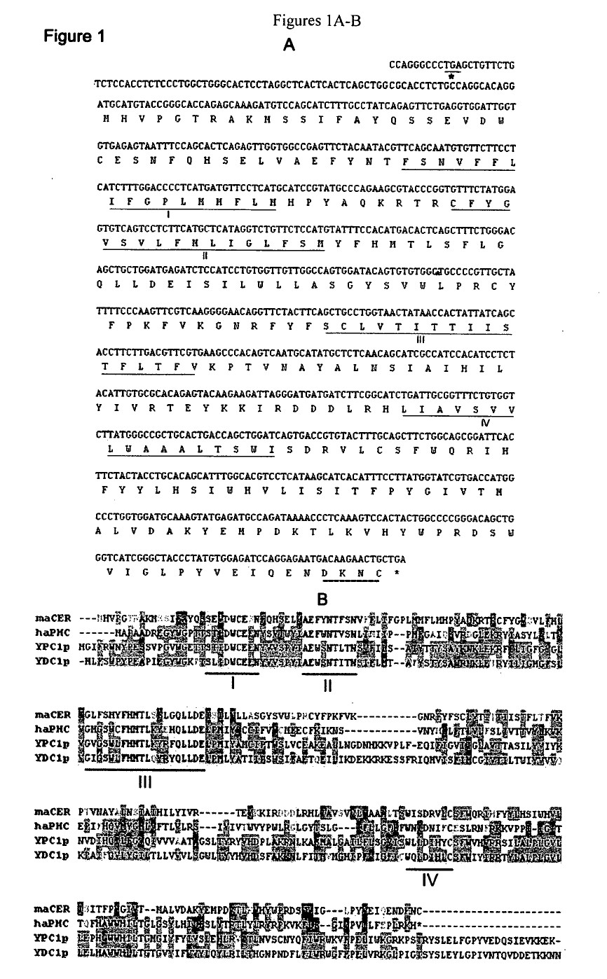

A BLAST search of EST databases in GenBank against haPHC revealed a mouse expressed sequence tag (EST) encoding a 270 amino acid putative protein distinct from but significantly homologous to haPHC. The coding sequence of the putative protein preceded by 5′ untranslated region (5′-UTR) was cloned from a mouse liver cDNA library by RACE as described above under “Experimental Procedures” (FIG. 1A). Sequencing analysis revealed an in-frame stop codon upstream of the first ATG codon of the coding region. The flanking sequence of the ATG codon matched with the Kozak consensus sequence, suggesting the authenticity of the predicted ORF. Sequence alignment showed that this putative protein exhibited 28% identity to both YPC1p and YDC1p, and 32% to haPHC (FIG. 1B), and that it contained several conserved regions shared by the alkaline ceramidases. Similar to the other alkaline ceramidases, this putative protein has five putative transmembrane domains as predicted by a pSORTII program (FIG. 1A). Based on the sequence similarity to the alkaline ceramidases, this mouse protein is postulated to be a new member of the alkaline ceramidase family, designated as the mouse alkaline ceramidase 1 (maCER1).

maCER1 is a Bona Fide Ceramidase that Hydrolyzes D-erythro-ceramide

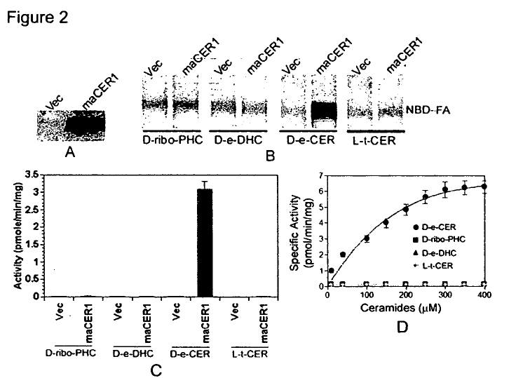

To investigate whether maCER1 is a bona fide ceramidase, it was tagged with the FLAG epitope and expressed in a yeast mutant (Δypc1ydc1) devoid of ceramidase activity as described above under “Experimental Procedures”. Microsomes were prepared from the Δypc1ydc1 strain transformed with the empty vector (the Vec strain) or the maCER1-expressing construct pYES2-FLAG-maCER1 (the maCER1 strain). Expression of the FLAG-tagged maCER1 was verified by Western blot analysis using anti-FLAG antibody. The FLAG-tagged maCER1 was associated with membranes isolated from the maCER1 strain, but not from the Vec strain (FIG. 2A). The microsomes were assayed for ceramidase activity using NBD-C₁₂-ceramides as substrates. As shown in FIGS. 2B and C, the microsomes of the maCER1 strain, but not the Vec strain, had a significant activity towards D-e-C₁₂-NBD-ceramide (100 μM). Neither the Vec nor maCER1 microsomes exhibited ceramidase activity towards D-e-C₁₂-NBD-dihydroceramide, D-ribo-C₁₂-NBD-phytoceramide, or L-t-C₁₂-NBD-ceramide, a stereoisomer of D-e-C₁₂-ceramide at the same concentration. The ceramidase activity toward -D-e-C₁₂-NBD-ceramide, but not the stereoisomers of D-e-C₁₂-NBD-ceramide, was increased in a dose dependent manner (FIG. 2D). These results indicate that maCER1 is a ceramidase with a highly restricted substrate specificity for the natural stereoisomer of ceramide with D-erythro-sphingosine, but not D-ribo-phytosphingosine or D-erythro-dihydrosphingosine as a backbone.

We have shown previously that the yeast alkaline phytoceramidase YPC1p has a considerable reverse activity. In order to determine whether maCER1 has such an activity, the same microsomal preparations were assayed for reverse activity using [³H]-palmitic acid and sphingosine, dihydrosphingosine, or phytosphingosine as substrates as described above under “Experimental Procedures” maCER1 had a minor reverse activity only when sphingosine was used, but not when phytosphingosine or dihydrosphingosine was used as substrate. These results suggest that maCER1 has a major ceramidase activity and only a very minor reverse activity.

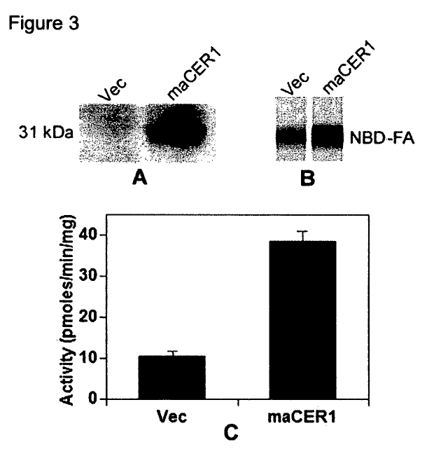

We further confirmed that maCER1 is a ceramidase by expressing it in mammalian cells. COS1 cells were transfected with the vector pcDNA3.1(+)-FLAG or the construct pcDNA3.1(+)-FLAG-maCER1 as described above under “Experimental Procedures”. Forty-eight hours after transfection, membrane fractions were prepared from the transfected cells as described above under Experimental Procedures. Proteins were extracted from the membrane fractions (microsomes) sedimented at 100K g, and were subjected to Western blot analysis for expression of the FLAG-tagged maCER1 using anti-FLAG antibody as described previously. The FLAG-maCER1 was detected in the microsomes prepared from the COS1 cells that were transfected with the expression construct (maCER1), but not with the empty vector (Vec) (FIG. 3A). The same microsomal preparations were assayed for ceramidase activity using D-e-C₁₂-NBD-ceramide as substrate. Ceramidase activity was increased 4-fold in the maCER1 microsomes compared to that in the Vec microsomes (FIGS. 3B and C). These results confirm that maCER1 is indeed a bona fide ceramidase, with a highly restricted substrate specificity for ceramide with sphingosine as a backbone.

maCER1 has an Alkaline pH Optimum and its Activity is Affected by Cations

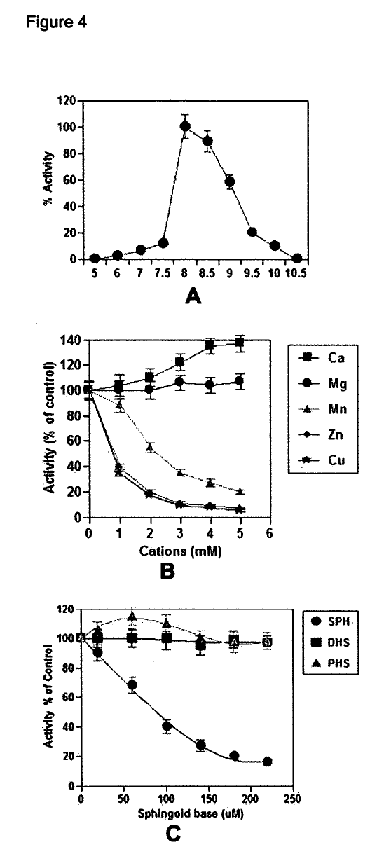

To evaluate biochemical properties of maCER1, first, we determined its pH optimum. Microsomes were isolated from the maCER1 yeast strain. Ceramidase activity in the microsomes was determined using D-e-C₁₂-NBD-ceramide as substrate at different pH in the presence of 5 mM Ca²⁺. As shown in FIG. 4A, maCER1 had a negligible activity at pH lower than 6.5 or higher than 10.5, a slight activity at pH between 6-7.5 or 9.5-10, and the highest activity around pH 8. These results indicate that, similar to other alkaline ceramidases, maCER1 displays an alkaline pH optimum.

Second, cation dependence of ceramidase activity of maCER1 was studied. Ceramidase activity of the maCER1-containing microsomes was determined at pH 8 in the presence of different cations with various concentrations. As shown in FIG. 4B, Ca²⁺ slightly activated ceramidase in a dose dependent manner, but Mn²⁺, Zn²⁺, and Cu²⁺ inhibited the activity in a dose-dependent manner, with a respective 10, 60, and 60% inhibition at 1 mM. Mg²⁺ had no effect at the same concentrations.

Finally, effects of sphingoid bases on maCER1 were investigated. As shown in FIG. 4C, sphingosine inhibited ceramidase activity in a dose-dependent manner. The IC₅₀ for the inhibition was approximately 80 μM. In contrast, neither phytosphingosine nor dihydrosphingosine in the same concentration range were inhibitory.

maCER1 is Highly Expressed in Skin

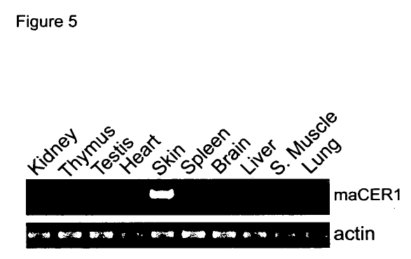

To determine tissue-specific expression of maCER1 mRNA, we performed Northern blot analysis on a Multiple Tissue mRNA blot (Clontech, Inc.) containing mRNA from mouse heart, brain, placenta, lung, liver, skeletal muscle, kidney, and pancreas. The Northern blot analysis failed to detect maCER1 mRNA, suggesting its low transcription levels in these major organs. Then RT-PCR analysis, a more sensitive approach, was performed on RNA isolated from a variety of organs as described under Experimental Procedures. Consistent with the Northern blot analysis, RT-PCR analysis demonstrated that maCER1 mRNA was expressed at a very low level in the aforementioned organs or tissues (FIG. 5). However, maCER1 was highly expressed in skin.

maCER1 is an ER Protein

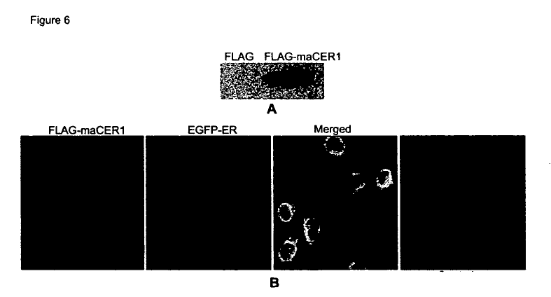

Similar to other members of the alkaline ceramidase, maCER1 contains a Golgi to ER retrieval signal sequence (FIG. 1A), suggesting that it may be localized to the ER. To test this, first we established Hela cell lines that stably express the FLAG tag (Vec) and the FLAG-tagged maCER1 (maCER1) respectively as described above under “Experimental Procedures”. Western blot analysis confirmed that the maCER1 stable cell line, but not the Vec cell line, expressed the FLAG-tagged maCER1, which was associated with membranes (FIG. 6A). The maCER1 stable cell line was transiently transfected with pEGFP-ER that directed the expression of EGFP (as a fluorescent ER marker) in the ER as described above under “Experimental Procedures”. After transfection, the maCER1 cells were probed by an anti-FLAG antibody followed by goat anti-mouse IgG conjugated with rhodamine. The Vec cell line (FLAG) exhibited a weak red fluorescent background whereas the maCER1 cell line (FLAG-maCER1) displayed a red fluorescent pattern of para-nuclear reticulum network, which completely overlapped with the green fluorescent pattern of the EGFP-ER marker (FIG. 6B), suggesting that the FLAG-maCER1 was localized to the ER.

maCER1 Has the Ability to Regulate Metabolism of Sphingolipids

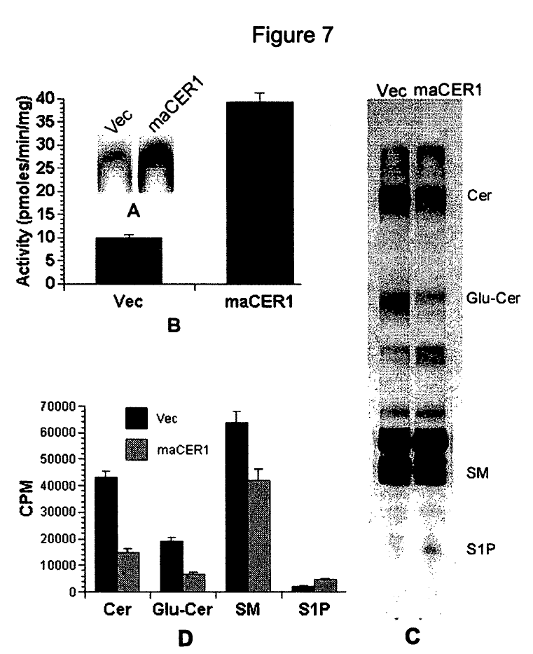

In vitro activity assays showed that the alkaline ceramidase activity in the maCER1 cell line was increased four-fold compared to that in the Vec cell line (FIGS. 7A and B). To determine whether the increased activity of the alkaline ceramidase had an effect on metabolism of sphingolipids in cells, metabolic labeling was performed. At 80% confluence, the Vec and maCER1 cell lines were labeled with [³H]-DHS as described above under “Experimental Procedures”. After being extracted from the labeled cells, total lipids were resolved by TLC. Radioactive bands corresponding to sphingolipids were detected by autoradiography. TLC analysis demonstrated that radio-labeled ceramide, glucosylceramide, and sphingomyelin were decreased three-, four-, and one and a half-fold, respectively, whereas radiolabled S1P was increased 2.5-fold in the maCER1 cell line, compared to the Vec cell line (FIGS. 7C and D). These results suggest that maCER1 has the ability to regulate metabolism of sphingolipids by controlling hydrolysis of ceramide in cells.

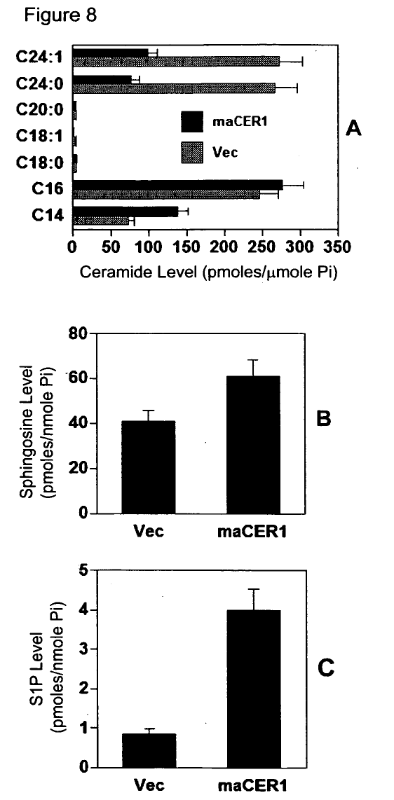

To determine whether maCER1 can regulate the cellular levels of ceramides, sphingosine (SPH), and S1P, lipids were extracted from 90% confluent cells grown in DMEM and were subjected to mass spectrometry analysis as described above under “Experimental Procedures”. As shown in FIG. 8A, the levels of D-e-C_{16:0}-ceramide were similar in both Vec and maCER1 cell lines, whereas those of C_{24:0} and C_{24:1}-ceramides were decreased 3 and 4-fold, respectively, in the maCER1 cell line, compared to the Vec cell line. Interestingly, the levels of D-e-C_{14:0}-ceramide were increased 2-fold in the maCER1 cell line compared to the Vec cell line. In the maCER1 cell line, the levels of sphingosine were slightly increased (FIG. 8B), but those of S1P were robustly increased (more than 7-fold) (FIG. 8C), compared to the Vec cell line. These results indicate that maCER1 is capable of regulating the levels of ceramides with very long fatty acyl chains and those of S1P.

Cloning of a Human Alkaline Ceramidase 1 (haCER1)

A BLAST search of databases in GenBank revealed an expressed sequence tag (EST) encoding a polypeptide highly homologous to the mouse alkaline ceramidase 1 (maCER1) (FIG. 1A). The coding sequence of this polypeptide was cloned from a human liver cDNA library by PCR using 5′ and 3′ primers corresponding to the start and stop regions, respectively, of the putative coding region as described under “Experimental Procedures”. Sequencing analysis showed that the cloned putative coding sequence is identical to the sequence reported in the EST database. In order to verify the putative translation initiation site, a 5′-untranslational region (5-UTR) upstream the putative coding sequence was cloned by 5′-RACE. This 5′-UTR contains an in-frame stop codon (underlined in FIG. 1A) proximal to the putative start codon, suggesting that the putative coding sequence is authentic. Protein sequence alignment demonstrated that the human alkaline ceramidase homologue has an 88% identity to maCER1 (FIG. 1B), indicating that it is the maCER1 orthologue, designated as the human alkaline ceramidase 1 (haCER1). haCER1 also exhibits a high degree of sequence similarity to other characterized alkaline ceramidases.

haCER1 is an Alkaline Ceramidase with a Substrate Specificity Towards Unsaturated Long Chain Ceramides

To experimentally verify that it is a bona fide alkaline ceramidase, haCER1 was expressed in the yeast mutant strain Δypc1Δydc1 that lacks the endogenous alkaline ceramidase activity due to the deletion of YPC1p and YDC1P. The coding sequence of haCER1 was cloned into a yeast vector pYES2-FLAG that we previously constructed. The resulting expression construct pYES2-FLAG-haCER1 or the empty vector pYES2-FLAG was transfected into the Δypc1Δydc1 cells. The resulting transformants were analyzed for the expression of the FLAG-tagged haCER1 by Western blot analysis using anti-FLAG antibody. As shown in FIG. 2A, the expression of the FLAG-tagged haCER1 was detected in microsomes isolated from yeast cells transformed with pYES2-FLAG-haCER1, but not with pYES2-FLAG. The microsomes were assayed for ceramidase activity using NBD-ceramides as substrates. The microsomes isolated from the FLAG-haCER1-expressing cells exhibited ceramidase activity towards NBD-ceramide, but not NBD-dihydroceramide or phytoceramide whereas the microsomes isolated from the vector-containing cells exhibited no ceramidase activity towards any NBD-ceramides (FIG. 2B). These results suggest that like the mouse ortholog maCER1, haCER1 is a bone fide alkaline ceramidase with substrate specificity for unsaturated ceramides.

### Discussion

Based on sequence similarity, maCER1, a mouse homologue of alkaline ceramidases was identified and cloned. In vitro and in vivo studies demonstrated that maCER1 is a ceramidase with a highly restricted substrate specificity, hydrolyzing mammalian D-erythro-ceramide exclusively, but not dihydroceramide (DHC) or phytoceramide (PHC). maCER1 mRNA was predominantly expressed in skin, and maCER1 was localized to the ER. Overexpression of maCER1 elevated ceramidase activity and caused a decrease in the incorporation of the radioactive precursor [³H]-DHS into ceramide and complex sphingolipids, but enhanced the diversion of [³H]-DHS to S1P. Mass measurement suggested that the increase in maCER1 activity caused a decrease in the cellular levels of both D-e-C_{24:0}-ceramide and D-e-C_{24:1} ceramide (but not other ceramides), and caused an increase in the levels of S1P. Taken together, these results suggest that maCER1 is a novel ceramidase potentially capable of regulating the levels of distinct ceramides, S1P, and complex sphingolipids.

We have identified and cloned several alkaline ceramidases from yeast, mice, and humans. Each enzyme appears to be responsible for the deacylation of a ceramide with a distinct sphingoid base backbone. The yeast alkaline ceramidases YPC1p and YDC1p deacylate phytoceramide and dihydroceramide, respectively (References 19 and 20). In vitro, the human alkaline phytoceramidase (haPHC) hydrolyzes phytoceramide preferentially (Reference 21), whereas maCER1 hydrolyzes ceramide specifically. In addition to maCER1, sequence analysis revealed two other murine alkaline ceramidase homologues. One differs from haPHC by a few amino acids, designated as maPHC, and the other, designated as maCER2, has a 56% identity to maCER1. Their substrate specificity is not yet known, but maPHC probably hydrolyzes phytoceramide preferentially based on its high homology to haPHC. This observation suggests that hydrolysis of ceramides with different sphingoid base backbones is independently regulated by a specific alkaline ceramidase.

Alkaline ceramidases with different substrate specificity may have distinct physiological functions by regulating metabolism of different ceramides. We previously demonstrated that deletion of the yeast dihydroceramidase YDC1p, but not phytoceramidase YPC1p exhibits a heat stress phenotype (Reference 19). In the mammalian system, ceramides with different structures seem to have distinct roles either in signaling processes or structural functions of membranes. For example, ceramide is a potent pro-apoptotic molecule to numerous cell types, whereas dihydroceramide is ineffective (Reference 1). On the other hand, phytoceramide has been shown to be more potent than ceramide in induction of apoptosis of neuroblastoma cells (SK-N-BE(2)c) (Reference 25). It is plausible that maCER1, which hydrolyzes very long chain unsaturated ceramide, may regulate cellular responses mediated by this ceramide species, but not by saturated (dihydroceramide) or hydroxylated (phytoceramide). As for structural functions, ceramide, dihydroceramide, and phytoceramide with the same acyl chain exhibit different physiochemical properties. It is expected that membranes composed of different levels of these ceramides may also have different physiochemical properties, which may be regulated in part by maCER1 through controlling the relative levels of ceramide versus dihydroceramide and/or phytoceramide.

Metabolism of ceramides in different cellular compartments is independently regulated by distinct ceramidases. The mouse and human acid ceramidases with a pH optimum of 4-5 are localized to the lysosomes (Reference 26), whereas the human neutral ceramidase with a pH optimum of 6-7 is localized to mitochondria (Reference 14). Similar to maCER1, both acid and neutral ceramidases also hydrolyze the sphingosine-containing ceramides although they may have a different preference for acyl chains from maCER1. It is unclear why hydrolysis of ceramides in different cellular compartments is executed by distinct ceramidases, which share no similarity in their primary sequences. Ceramides with different acyl chains have been found in mammalian cells. Different cellular compartments may contain distinct pools of ceramide(s), metabolism of which may require specific ceramidases. Another possibility is that ceramides in different cellular compartments may have different physiological functions, therefore their metabolism needs to be independently regulated by distinct enzymes.

maCER1 appears to be predominantly expressed in skin, suggesting that it may have a specific role in skin function. It has been shown that extracellular ceramides and complex sphingolipids play important roles in forming the permeability barrier of skin (Reference 24). Macheleidt et al (Reference 27) showed that very long chain ceramides are significantly reduced in the epidermis of atopic dermatitis patients, suggesting that the very long chain ceramides have an important role in regulating the permeability barrier function. maCER1 may have a role in regulating the barrier function by regulating the levels of very long chain ceramides in skin. Intracellular ceramides and glycosylsphingolipids also have a role in regulating proliferation, differentiation, and apoptosis of epidermal keratinocytes (References 28 and 29). Uchida et al (Reference 29) showed that treatment of cultured human keratinocytes with bacterial sphingomyelinase, which releases ceramide from sphingomyelin in plasma membranes, inhibits cell proliferation, suggesting that ceramide may regulate proliferation of skin cells. maCER1 may regulate proliferation of keratinocytes by regulating the levels of ceramide. Uchida and his colleagues further showed that treatment of keratinocytes with PDMP, an inhibitor of glucosylceramide synthase, also inhibits cell proliferation, suggesting that gluocsylceramide or/and other gylcosylsphingolipids may have a role in regulating skin cell proliferation. We demonstrate that overexpression of maCER1 decreases synthesis of glucosylceramide, suggesting that maCER1 may regulate proliferation of keratinocytes by regulating biosynthesis of complex sphingolipids. The cloning of maCER1 provides an important molecular tool to dissect the roles of ceramides and complex sphingolipids in skin functions.

maCER1 mRNA is expressed at a very low level in organs and tissues other than skin, suggesting that its expression is tightly regulated. maCER1 is inhibited by sphingosine, one of its products, indicating that maCER1 may also be regulated at the post-translational level. Moreover, PROSITE motif search revealed that maCER1 contains three putative protein kinase C phosphorylation sites, two casein kinase II phosphorylation sites, and one tyrosine phophorylation site, suggesting that activity of this enzyme in cells may be regulated by phosphorylation. Coroneos et al (Reference 30) demonstrated that pretreating cells with an inhibitor of protein tyrosine phosphatase (sodium vanadate) augmented the stimulation of alkaline ceramidase activity by PDGF in mesangial cells, suggesting that tyrosine phosphorylation is involved in the activation of alkaline ceramidase(s). It remains to be determined whether maCER1 is regulated in response to PDGF.

In conclusion, we have identified or discovered, isolated, and cloned new human and mouse alkaline ceramidases. These ceramidases have the most stringent substrate specificity among the recently identified ceramidases. Their tissue specific expression and cellular localization is distinct from acid and neutral ceramidases. In particular, maCER1 is the first ceramidase shown to regulate the levels of distinct ceramides and S1P in cells. In conjunction with other alkaline ceramidases, the cloning of maCER1 and haCER1 will enable us to distinguish physiological roles of distinct ceramides and their derivatives.

The maCER1 cDNA and its encoded protein sequences were deposited with GenBank of GenBank Submissions, National Center for Biotechnology Information, National Library of Medicine, Bldg. 38A, Room 8N-803, Bethesda, Md. 20894 (USA) (www.ncbi.nlm.nih.gov) on Feb. 8, 2001, under Accession Numbers AF347023 and ML83821, respectively, which are hereby incorporated herein in their entirety by reference.

The haCER1 cDNA and its encoded protein sequences were deposited with GenBank of GenBank Submissions, National Center for Biotechnology Information, National Library of Medicine, Bldg. 38A, Room 8N-803, Bethesda, Md. 20894 (USA) (www.ncbi.nlm.nih.gov) on Feb. 8, 2001, under Accession Numbers AF347024 and ML83822, respectively, which are hereby incorporated herein in their entirety by reference.

While this invention has been described as having preferred sequences, ranges, steps, materials, structures, features, and/or designs, it is understood that it is capable of further modifications, uses and/or adaptations of the invention following in general the principle of the invention, and including such departures from the present disclosure as those come within the known or customary practice in the art to which the invention pertains, and as may be applied to the central features hereinbefore set forth, and fall within the scope of the invention and of the limits of the appended claims.

The following references, and those cited or discussed herein, are all hereby incorporated herein in their entirety by reference.
1. Hannun, Y. A., and Obeid, L. M. (2002) J Biol Chem 277, 25847-25850.
2. Merrill, A. H., Jr. (2002) J Biol Chem 277, 25843-25846.
3. Spiegel, S., and Milstien, S. (2002) J Biol Chem 277, 25851-25854.
4. el Bawab, S., Mao, C., Obeid, L. M., and Hannun, Y. A. (2002) Subcell Biochem 36, 187-205.
5. Pyne, S. (2002) Subcell Biochem 36, 245-268.
6. Brown, D. A., and London, E. (2000) J Biol Chem 275, 17221-17224.
7. van Meer, G., and Lisman, Q. (2002) J Biol Chem 277, 25855-25858.
8. Ferlinz, K., Kopal, G., Bernardo, K., Linke, T., Bar, J., Breiden, B., Neumann, U., Lang, F., Schuchman, E. H., and Sandhoff, K. (2001) J Biol Chem 276, 35352-35360.
9. Abenoza, P., and Sibley, R. K. (1987) Ultrastruct Pathol 11,397-403.
10. Li, C. M., Park, J. H., Simonaro, C. M., He, X., Gordon, R. E., Friedman, A. H., Ehleiter, D., Paris, F., Manova, K., Hepbiloikler, S., Fuks, Z., Sandhoff, K., Kolesnick, R., and Schuchman, E. H. (2002) Genomics 79, 218-224.
11. Okino, N., Ichinose, S., Omori, A., lmayama, S., Nakamura, T., and Ito, M. (1999) J Biol Chem 274, 36616-36622.
12. Tani, M., Okino, N., Mori, K., Tanigawa, T., Izu, H., and Ito, M. (2000) J Biol Chem 275, 11229-11234.
13. Mitsutake, S., Tani, M., Okino, N., Mori, K., Ichinose, S., Omori, A., lida, H., Nakamura, T., and Ito, M. (2001) J Biol Chem 276, 26249-26259.
14. El Bawab, S., Roddy, P., Qian, T., Bielawska, A., Lemasters, J. J., and Hannun, Y. A. (2000) J Biol Chem 275, 21508-21513.
15. Tani, M., Okino, N., Mitsutake, S., Tanigawa, T., Izu, H., and Ito, M. (2000) J Biol Chem 275, 3462-3468.
16. Kita, K., Okino, N., and Ito, M. (2000) Biochim Biophys Acta 1485, 111-120.
17. El Bawab, S., Birbes, H., Roddy, P., Szulc, Z. M., Bielawska, Z. M., and Hannun, Y. A. (2001) J Biol Chem 8, 8
18. Romiti, E., Meacci, E., Tani, M., Nuti, F., Farnararo, M., Ito, M., and Bruni, P. (2000) Biochem Biophys Res Commun 275, 746-751.
19. Mao, C., Xu, R., Bielawska, A., Szulc, Z. M., and Obeid, L. M. (2000) J Biol Chem 275, 31369-31378.
20. Mao, C., Xu, R., Bielawska, A., and Obeid, L. M. (2000) J Biol Chem 275, 6876-6884.
21. Mao, C., Xu, R., Szulc, Z. M., Bielawska, A., Galadari, S. H., and Obeid, L. M. (2001) J Biol Chem 16,16
22. Okazaki, T., Bielawska, A., Domae, N., Bell, R. M., and Hannun, Y. A. (1994) J Biol Chem 269, 4070-4077.
23. Marchesini, S., Preti, A., Aleo, M. F., Casella, A., Dagan, A., and Gatt, S. (1990) Chem Phys Lipids 53, 165-175.
24. Doering, T., Brade, H., and Sandhoff, K. (2002) J Lipid Res 43, 1727-1733.
25. Hwang, O., Kim, G., Jang, Y. J., Kim, S. W., Choi, G., Choi, H. J., Jeon, S. Y., Lee, D. G., and Lee, J. D. (2001) Mol Pharmacol 59, 1249-1255.
26. Koch, J., Gartner, S., Li, C. M., Quintern, L. E., Bernardo, K., Levran, O., Schnabel, D., Desnick, R. J., Schuchman, E. H., and Sandhoff, K. (1996) J Biol Chem 271, 33110-33115.
27. Macheleidt, O., Kaiser, H. W., and Sandhoff, K. (2002) J Invest Dermatol 119, 166-173.
28. Raisova, M., GoItz, G., Bektas, M., Bielawska, A., Riebeling, C., Hossini, A. M., Eberle, J., Hannun, Y. A., Orfanos, C. E., and Geilen, C. C. (2002) FEBS Lett 516, 47-52.
29. Uchida, Y., Murata, S., Schmuth, M., Behne, M. J., Lee, J. D., Ichikawa, S., Elias, P. M., Hirabayashi, Y., and Holleran, W. M. (2002) J Lipid Res 43, 1293-1302.
30. Coroneos, E., Martinez, M., McKenna, S., and Kester, M. (1995) J Biol Chem 270, 23305-23309.

What is claimed is:

1. An isolated nucleotide sequence as set forth in SEQ ID NO: 1.

2. The sequence of claim 1, which is substantially purified.

3. The sequence of claim 1, which is substantially biologically purified.

4. A cloned nucleotide sequence as set forth in SEQ ID NO: 1.

5. A nucleotide sequence encoding mouse alkaline ceramidase 1 (maCER1).

6. The sequence of claim 5, wherein the ceramidase is expressed in skin and hydrolyzes D-erythro-ceramide.

7. The sequence of claim 5, wherein the ceramidase possesses the ability to regulate the level of a long chain ceramide in mammalian cells.

8. The sequence of claim 5, wherein the ceramidase possesses the ability to regulate a complex sphingolipid in mammalian cells.

9. The sequence of claim 5, wherein the sequence comprises the sequence as set forth in SEQ ID NO:1.

10. The sequence of claim 5, wherein the ceramidase has a substrate specificity for the natural stereoisomer of ceramide with D-erythro-sphingosine as a backbone.

11. The sequence of claim 5, wherein the ceramidase is activated by a cation.

12. The sequence of claim 11, wherein the cation comprises Ca²⁺.

13. The sequence of claim 5, wherein the ceramidase is inhibited by at least one cation selected from the group consisting of Mn²⁺, Zn²⁺, and Cu²⁺.

14. The sequence of claim 5, wherein the ceramidase is inhibited by sphingosine.

15. The sequence of claim 5, wherein the ceramidase is expressed in skin cells.

16. The sequence of claim 5, wherein the ceramidase is localized to endoplasmic reticulum.

17. A vector comprising the nucleotide sequence of claim 9.

18. A plasmid comprising the nucleotide sequence of claim 9.

19. An expression construct comprising the nucleotide sequence of claim 9.

20. A probe comprising the nucleotide sequence of claim 9.

21. A substantially purified isolated nucleotide sequence encoding a mouse alkaline ceramidase having the ability to hydrolyze mammalian D-erythro-ceramide.

22. The sequence of claim 21, wherein the ceramidase further possesses the ability to hydrolyze a complex sphinogolipid.

23. The sequence of claim 21, wherein the sequence comprises the sequence as set forth in SEQ ID NO: 1.

24. An isolated mouse alkaline ceramidase having the ability to hydrolyze mammalian D-erythro-ceramide.

25. The ceramidase of claim 24, which is substantially purified.

26. The ceramidase of claim 24, which is substantially biologically purified.

27. A recombinant mouse alkaline ceramidase having the ability to hydrolyze mammalian D-erythro-ceramide.

28. A normaturally occurring analogue of the mouse alkaline ceramidase 1 (maCER1).

29. An isolated nucleotide sequence as set forth in FIG. 1A.

30. An isolated nucleotide sequence deposited with GenBank (www.ncbi.nlm.nih.gov), with an Accession Number AF347023.

31. An isolated nucleotide sequence as set forth in SEQ ID NO: 2.

32. The sequence of claim 31, which is substantially purified.

33. The sequence of claim 31, which is substantially biologically purified.

34. A cloned nucleotide sequence as set forth in SEQ ID NO: 2.

35. A nucleotide sequence encoding human alkaline ceramidase 1 (haCER1).

36. The sequence of claim 35, wherein the ceramidase is expressed in skin and hydrolyzes D-erythro-ceramide.

37. The sequence of claim 35, wherein the ceramidase possesses the ability to regulate the level of a long chain ceramide in mammalian cells.

38. The sequence of claim 35, wherein the ceramidase possesses the ability to regulate a complex sphingolipid in mammalian cells.

39. The sequence of claim 35, wherein the sequence comprises the sequence as set forth in SEQ ID NO: 2.

40. The sequence of claim 35, wherein the ceramidase has a substrate specificity for the natural stereoisomer of ceramide with D-erythro-sphingosine as a backbone.

41. The sequence of claim 35, wherein the ceramidase is activated by a cation.

42. The sequence of claim 41, wherein the cation comprises Ca²⁺.

43. The sequence of claim 35, wherein the ceramidase is inhibited by at least one cation selected from the group consisting of Mn²⁺, Zn²⁺, and Cu²⁺.

44. The sequence of claim 35, wherein the ceramidase is inhibited by sphingosine.

45. The sequence of claim 35, wherein the ceramidase is expressed in skin cells.

46. The sequence of claim 35, wherein the ceramidase is localized to endoplasmic reticulum.

47. A vector comprising the nucleotide sequence of claim 39.

48. A plasmid comprising the nucleotide sequence of claim 39.

49. An expression construct comprising the nucleotide sequence of claim 39.

50. A probe comprising the nucleotide sequence of claim 39.

51. A substantially purified isolated nucleotide sequence encoding a human alkaline ceramidase having the ability to hydrolyze mammalian D-erythro-ceramide.

52. The sequence of claim 51, wherein the ceramidase further possesses the ability to hydrolyze a complex sphinogolipid.

53. The sequence of claim 51, wherein the sequence comprises the sequence as set forth in SEQ ID NO: 2.

54. An isolated human alkaline ceramidase having the ability to hydrolyze mammalian D-erythro-ceramide.

55. The ceramidase of claim 54, which is substantially purified.

56. The ceramidase of claim 54, which is substantially biologically purified.

57. A recombinant mouse alkaline ceramidase having the ability to hydrolyze mammalian D-erythro-ceramide.

58. A nonnaturally occurring analogue of the human alkaline ceramidase 1 (haCER1).

59. An isolated nucleotide sequence deposited with GenBank (www.ncbi.nlm.nih.gov), with an Accession Number AF347024.

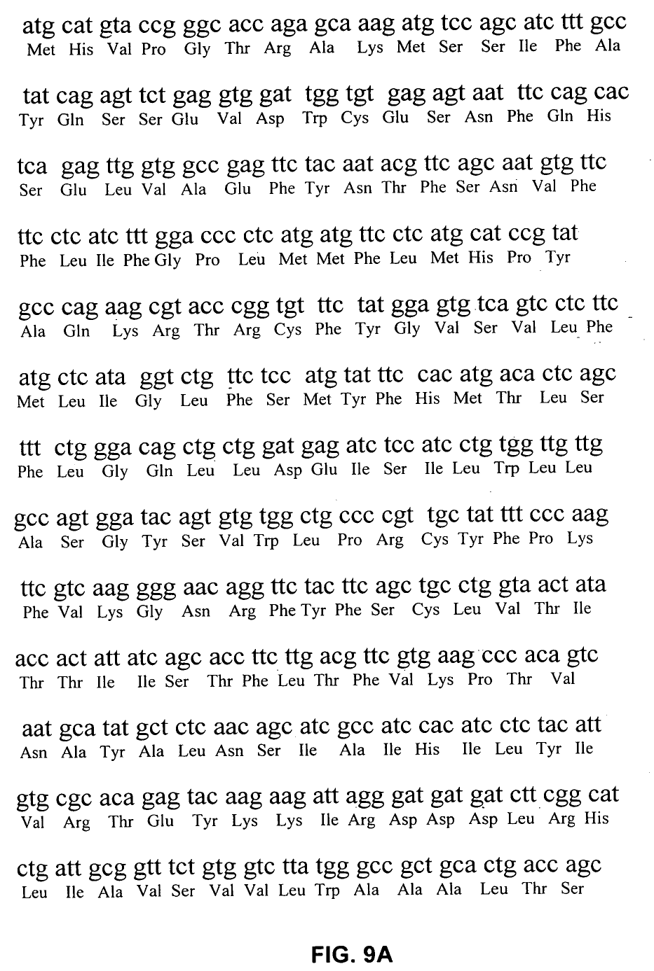

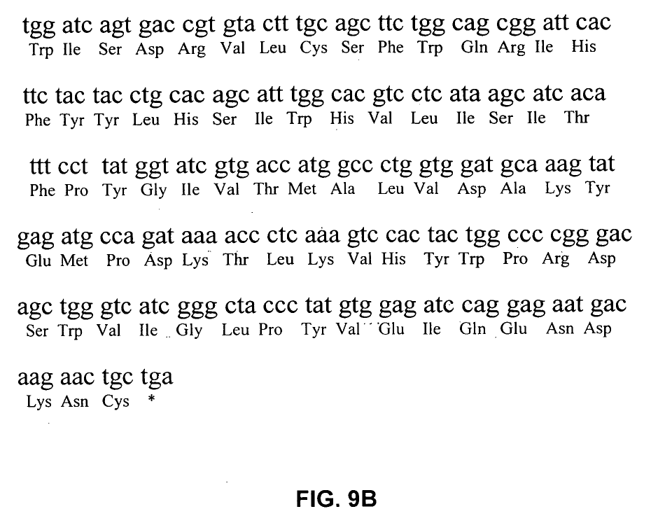

60. An isolated nucleotide sequence as set forth in FIGS. 9A-9B.

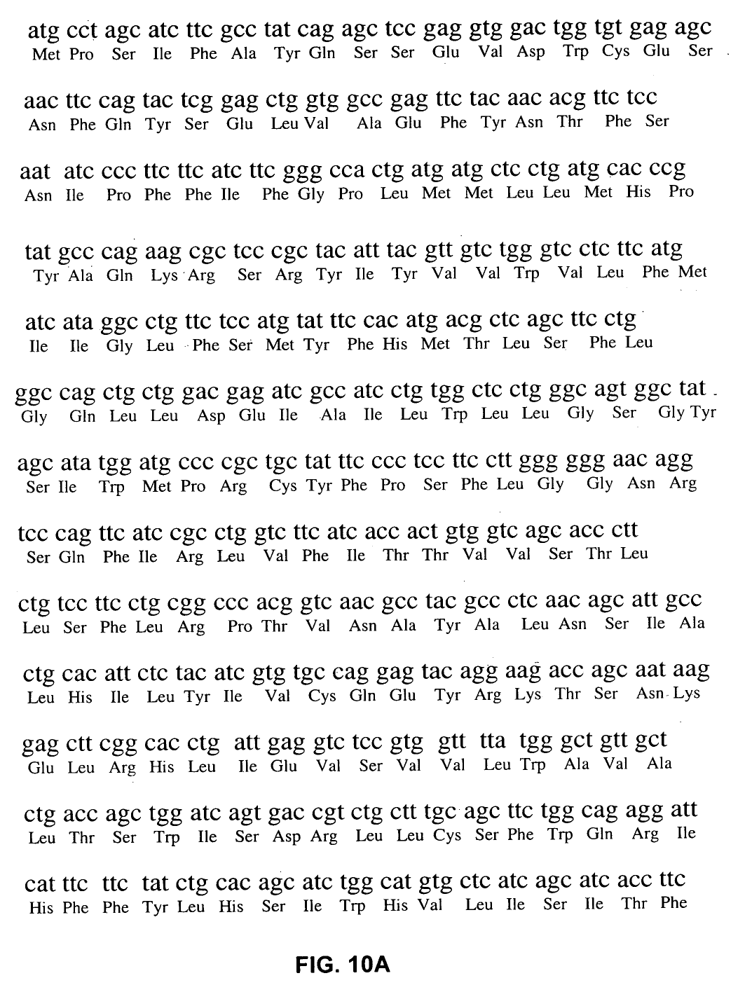

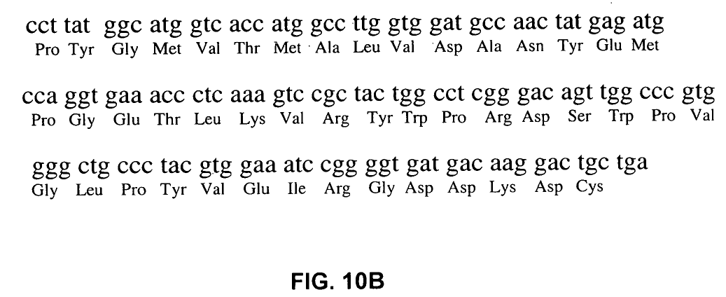

61. An isolated nucleotide sequence as set forth in FIGS. 10A-10B.

62. A recombinant protein sequence deposited with GenBank (www.ncbi.nlm.nih.gov) with an Accession Number AAL83821.

63. A recombinant protein sequence deposited with GenBank (www.ncbi.nlm.nih.gov) with an Accession Number AAL83822.
